SEQUENCE LISTING (39 sequences)

SEQ ID NO: 1 - protein, 933 aa, Staphylococcus aureus
MNMKKKEKHAIRKKSIGVASVLVGTLIGFGLLSSKEADASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTFNDTNTVSSVNSPQNSTNAENVSTTQDTSTEATPSNNESAPQSTDASNKDVVNQAVNTSAPRMRAFSLAAVAADAPAAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPEDSDSDPGSDSGSDSNSDSGSDSGSDSTSDSGSDSASDSDSASDSDSASDSDSASDSDSASDSDSDNDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSASDSDSDSDSDSDSDSDSDSDSDSDSDSDSDSDSDSDSDSESDSDSESDSDSDSDSDSDSDSDSDSDSDSASDSDSGSDSDSSSDSDSESDSNSDSESGSNNNVVPPNSPKNGTNASNKNEAKDSKEPLPDTGSEDEANTSLIWGLLASIGSLLLFRRKKENKDKK

SEQ ID NO: 2 - DNA, 3499 bp, Staphylococcus aureus
ggtaccataaattacacatctgcttttgaaaaaatatgatttcaagctaggattacattaggtagagttcatattaataataaaaaatgtttgcaatcaaatcgtacgttgtcgtttgtaattcttaaaatagcaataaataaaatgtttgttagtaaagtattattgtggataataaaatatcgatacaaattaattgctataatgcaattttagtgtataattccattaacagagattaaatatatctttaaagggtatatagttaatataaaatgactttttaaaaagagggaataaaatgaatatgaagaaaaaagaaaaacacgcaattcggaaaaaatcgattggcgtggcttcagtgcttgtaggtacgttaatcggttttggactactcagcagtaaagaagcagatgcaagtgaaaatagtgttacgcaatctgatagcgcaagtaacgaaagcaaaagtaatgattcaagtagcgttagtgctgcacctaaaacagacgacacaaacgtgagtgatactaaaacatcgtcaaacactaataatggcgaaacgagtgtggcgcaaaatccagcacaacaggaaacgacacaatcatcatcaacaaatgcaactacggaagaaacgccggtaactggtgaagctactactacgacaacgaatcaagctaatacaccggcaacaactcaatcaagcaatacaaatgcggaggaattagtgaatcaaacaagtaatgaaacgacttttaatgatactaatacagtatcatctgtaaattcacctcaaaattctacaaatgcggaaaatgtttcaacaacgcaagatacttcaactgaagcaacaccttcaaacaatgaatcagctccacagagtacagatgcaagtaataaagatgtagttaatcaagcggttaatacaagtgcgcctagaatgagagcatttagtttagcggcagtagctgcagatgcaccggcagctggcacagatattacgaatcagttgacgaatgtgacagttggtattgactctggtacgactgtgtatccgcaccaagcaggttatgtcaaactgaattatggtttttcagtgcctaattctgctgttaaaggtgacacattcaaaataactgtacctaaagaattaaacttaaatggtgtaacttcaactgctaaagtgccaccaattatggctggagatcaagtattggcaaatggtgtaatcgatagtgatggtaatgttatttatacatttacagactatgtaaatactaaagatgatgtaaaagcaactttgaccatgcccgcttatattgaccctgaaaatgttaaaaagacaggtaatgtgacattggctactggcataggtagtacaacagcaaacaaaacagtattagtagattatgaaaaatatggtaagttttataacttatctattaaaggtacaattgaccaaatcgataaaacaaataatacgtatcgtcagacaatttatgtcaatccaagtggagataacgttattgcgccggttttaacaggtaatttaaaaccaaatacggatagtaatgcattaatagatcagcaaaatacaagtattaaagtatataaagtagataatgcagctgatttatctgaaagttactttgtgaatccagaaaactttgaggatgtcactaatagtgtgaatattacattcccaaatccaaatcaatataaagtagagtttaatacgcctgatgatcaaattacaacaccgtatatagtagttgttaatggtcatattgatccgaatagcaaaggtgatttagctttacgttcaactttatatgggtataactcgaatataatttggcgctctatgtcatgggacaacgaagtagcatttaataacggatcaggttctggtgacggtatcgataaaccagttgttcctgaacaacctgatgagcctggtgaaattgaaccaattccagaggattcagattctgacccaggttcagattctggcagcgattctaattcagatagcggttcagattcgggtagtgattctacatcagatagtggttcagattcagcgagtgattcagattcagcaagtgattcagactcagcgagtgattcagattcagcaagcgattccgactcagcgagcgattccgactcagacaatgactcggattcagatagcgattctgactcagacagtgactcagattccgacagtgactcagattcagatagcgattctgactcagacagtgactcagattcagatagcgattcagattcagatagcgattcagattccgacagtgattccgactcagacagcgattctgactccgacagtgattccgactcagacagcgattcagattccgacagtgattccgactcagatagcgattccgactcagatagcgactcagattcagacagcgattcagattcagacagcgattcagattcagatagcgattcagattccgacagtgactcagattccgacagtgactcggattcagatagcgattcagattccgacagtgactcagattccgacagtgactcagactcagacagtgattcggattcagcgagtgattcggattcagatagtgattccgactccgacagtgactcggattcagatagcgactcagactcggatagcgactcggattcagatagcgattcggactcagatagcgattcagaatcagacagcgattcagaatcagacagcgattcagattcagacagcgactcagacagtgactcagattcagatagtgactcggattcagcgagtgattcagactcaggtagtgactccgattcatcaagtgattccgactcagaaagtgattcaaatagcgattccgagtcaggttctaacaataatgtagttccgcctaattcacctaaaaatggtactaatgcttctaataaaaatgaggctaaagatagtaaagaaccattaccagatacaggttctgaagatgaagcaaatacgtcactaatttggggattattagcatcaataggttcattactacttttcagaagaaaaaaagaaaataaagataagaaataagtaataatgatattaaattaatcatatgattcatgaagaagccaccttaaaaggtgcttcttttacttggattttccaaatatattgtttgaatataattaataattaattcatcaacagttaattattttaaaaaggtagatgttatataatttggcttggcgaaaaaatagggtgtaaggtaggttgttaattagggaaaattaaggagaaaatacagttgaaaaataaattgctagttttatcattgggagcattatgtgtatcacaaatttgggaaagtaatcgtgcgagtgcagtggtttctggggagaagaatccatatgtatctgagtcgttgaaactgactaataataaaaataaatctagaacagtagaagagtataagaaaagctt

SEQ ID NO: 3 - DNA, 2973 bp, Staphylococcus aureus
ttatgctttgtgattctttttatttctgcgtaataatgctaaacctagaatgctgaataatccgccgaacaacatacctttgtttgttgattcttctccacctgtttcaggtagttcagatttcttagattgtggttttttagttggtgccactgctttaaccttttcattgatttcaataacaggtgttactactttaccttgttccactggtttagaaggctttttaggttcttctttggcaggtggtactggtttaccaggttcagctggtacctctggtgttggcggtgttggagtttctggctcactcggcacttctggtgtcggtggtgttggtgtttccggctcacttggtacttctggtgttggtggcgttggtgtttccggctcacttggtacttctggtgtcggtggcgttggtggcacgattggaggtgttgtatcttcttcaatcgtttgttgaccttcattttggccgcttacttttggaagtgtatcttcttcaaagtcaacactattgtgtccaccgaattgataacttggtttatctttatttgtatcttcttcaataatttcagtgtgcttattgaatccgtgaatatgtggcacactgtcgaagtcgatatcaatgatgttaccgccatgttcatacttaggtttgtctttttctgtatcttcctcgaatgactgattacctttattttgaccatgaatttgaggtacactatcaaaatcgatatctacgatattgccaccttgttcatatttaggtttgtcttcttctgtgtcttcctcgaatgactggttaccgctattttggccaccttcataacctaattcactcttaatatcaacgtggctattttcttcgatttcttcaatcacgtcataattcccgtgaccattttcagttcctaaaccagaatgagaaatatgatgattgtttttagtaatttcctcgactggtccttgtgcttgaccatgctcttcaggtaattcatccactaattcaatcagattactttcagttgtatattctttcgtatcttcaactgttgtatgatcgctcactgcgccagttacaataccttttgtagactcttcgtcaaattcaactaagttagactcagtagtaacctgaccaccacctgggtttgtatcttcttcatattcaacaacatcagcgtgatgttttgaattttcatgtgtagattcttcaaagtcaattggatttgattcctcagaggactcagtgtatcctccaacgtgacctgcttcgctatccacagcagtatggtaatcgatatcaatagctgatgaatccgtttcttctattgtttcaatgtatccatcaacatatccacctccaccatctatagctgtgtggtaatcaatgtcaagagttgatgaatcatattcctcttcaacagtagttactaaattcttatcatattgacctgtaagagtttctttaattgtatcttctttatattcaaatttattattttgaataatcggaccatttttctcatttccgttcgctttattactgtataaaactaaaccattatcccaagttaaggtatatcctctatcataataatacttataaagttgctctggatgtcctaccatttgtgttctaaaatcaacttcatcagtaccatttaaatactctccatcatagtgaacaacataagttttatctagattttctatattcaatgaatagcttccattattttgtaaattcaaattcccactcatattacttgtgacttctttaaatttagaagtatctgtcgtatttgcatatacactcttcgctatgtcttcattattacccaagtattcaaatatcctaacttttggttgatttccattctgattactacctttcattaaagttccagtaacagtcacacttgtcgttttaccattattaggtttaataaatgcaacatgcgaaaatctattattcgctttattaaatgtctcaatcgatccatttaaattggcataataattcccaataccatctttatatttaacatctaattcctttgaagtttgttcttcatttagtgttgaagttatagtttgatttccattagtttgtacagttttaggatcaataaataaattaatttctagttcagccgttacatcaaccttatcttcaatatcatttgtaaatgtatatctaatctttccaccttctaaaacttcacctgtcgccattacgactgaaccatttttaatttctggtacttttctagcagttgatacgccatgcgtatttacattatttgataaagtaaagtcaaagtagtcaccttgatgtaaaccattctcaaatttcaacttatattttagtaccgctcgttgtcctgcatgaggttctactttatttgtattgttatgcccctcaatagaaccaatttctactgtaactttacttgttacatctgtacccgtttccactttcgcgttactagcttccttagcttccgctacatctgctgatcttgtcacacgtggcttactttctgatgccgttcttggctgtgccacttcaacttgtgtttctgcgacttgattttgtgtagcctttttaggtgttaaatctacttgtctttgatctccgctattgtcttgagattgtgttgtttccttaacttgaggtttcgcttcttccttaactacctcttctttaactgtttctatatttgctggttgtgcagtttgtggtgcttgtactgcttttggtgcttcttcagttgttacttgtgttgcgtttgacggttgttctgttactgttgcgttatatgattgagtttcttctatatgattaacgttagttgcagttgtttgtgtttcacttgttttattatcagtagctgaattcccattttcttctactgtagttgtcttttgttctgatgctgcagcttctttgtcttgtcccat

SEQ ID NO: 4 - protein, 990 aa, Staphylococcus aureus
MGQDKEAAASEQKTTTVEENGNSATDNKTSETQTTATNVNHIEETQSYNATVTEQPSNATQVTTEEAPKAVQAPQTAQPANIETVKEEVVKEEAKPQVKETTQSQDNSGDQRQVDLTPKKATQNQVAETQVEVAQPRTASESKPRVTRSADVAEAKEASNAKVETGTDVTSKVTVEIGSIEGHNNTNKVEPHAGQRAVLKYKLKFENGLHQGDYFDFTLSNNVNTHGVSTARKVPEIKNGSVVMATGEVLEGGKIRYTFTNDIEDKVDVTAELEINLFIDPKTVQTNGNQTITSTLNEEQTSKELDVKYKDGIGNYYANLNGSIETFNKANNRFSHVAFIKPNNGKTTSVTVTGTLMKGSNQNGNQPKVRIFEYLGNNEDIAKSVYANTTDTSKFKEVTSNMSGNLNLQNNGSYSLNIENLDKTYVVHYDGEYLNGTDEVDFRTQMVGHPEQLYKYYYDRGYTLTWDNGLVLYSNKANGNEKNGPIIQNNKFEYKEDTIKETLTGQYDKNLVTTVEEEYDSSTLDIDYHTAIDGGGGYVDGYIETIEETDSSAIDIDYHTAVDSEAGHVGGYTESSEESNPIDFEESTHENSKHHADVVEYEEDTNPGGGQVTTESNLVEFDEESTKGIVTGAVSDHTTVEDTKEYTTESNLIELVDELPEEHGQAQGPVEEITKNNHHISHSGLGTENGHGNYDVIEEIEENSHVDIKSELGYEGGQNSGNQSFEEDTEEDKPKYEQGGNIVDIDFDSVPQIHGQNKGNQSFEEDTEKDKPKYEHGGNIIDIDFDSVPHIHGFNKHTEIIEEDTNKDKPSYQFGGHNSVDFEEDTLPKVSGQNEGQQTIEEDTTPPIVPPTPPTPEVPSEPETPTPPTPEVPSEPETPTPPTPEVPSEPETPTPPTPEVPAEPGKPVPPAKEEPKKPSKPVEQGKVVTPVIEINEKVKAVAPTKKPQSKKSELPETGGEESTNKGMLFGGLFSILGLALLRRNKKNHKA

SEQ ID NO: 5 - protein, 16 aa, Lactobacillus lactis
SENSVTQSDSASNESK

SEQ ID NO: 6 - protein, 12 aa, Lactobacillus lactis
SNDSSSVSAAPK

SEQ ID NO: 7 - protein, 13 aa, Lactobacillus lactis
DVVNQAVNTSAPR

SEQ ID NO: 8 - protein, 13 aa, Lactobacillus lactis
LNYGFSVPNSAVK

SEQ ID NO: 9 - protein, 12 aa, Staphylococcus aureus
ELNLNGVTSTAK

SEQ ID NO: 10 - protein, 15 aa, Lactobacillus lactis
ATLTMPAYIDPENVK

SEQ ID NO: 11 - protein, 17 aa, Lactobacillus lactis
TGNVTLATGIGSTTANK

SEQ ID NO: 12 - protein, 7 aa, Lactobacillus lactis
FYNLSIK

SEQ ID NO: 13 - protein, 38 aa, Lactobacillus lactis
QTIYVNPSGDNVIAPVLTGNLKPNTDSNALIDQQNTSI

SEQ ID NO: 14 - protein, 36 aa, Staphylococcus aureus
VDNAADLSESYFVNPENFEDVTNSVNITFPNPNQYK

SEQ ID NO: 15 - protein, 27 aa, Lactobacillus lactis
VEFNTPDDQITTPYIVVVNGHIDPNSK

SEQ ID NO: 16 - protein, 41 aa, Lactobacillus lactis
TSETQTTATNVNHIEETQSYNATVTEQPSNATQVTTEEAPK

SEQ ID NO: 17 - protein, 17 aa, Lactobacillus lactis
AVQAPQTAQPANIETVK

SEQ ID NO: 18 - protein, 22 aa, Staphylococcus aureus
AVQAPQTAQPANIETVKEEVVK

SEQ ID NO: 19 - protein, 13 aa, Staphylococcus aureus
EEVVKEEAKPQVK

SEQ ID NO: 20 - protein, 18 aa, Lactobacillus lactis
KATQNQVAETQVEVAQPR

SEQ ID NO: 21 - protein, 17 aa, Lactobacillus lactis
ATQNQVAETQVEVAQPR

SEQ ID NO: 22 - protein, 16 aa, Lactobacillus lactis
VTVEIGSIEGHNNTNK

SEQ ID NO: 23 - protein, 28 aa, Staphylococcus aureus
FENGLHQGDYFDFTLSNNVNTHGVSTAR

SEQ ID NO: 24 - protein, 16 aa, Staphylococcus aureus
NGSVVMATGEVLEGGK

SEQ ID NO: 25 - protein, 10 aa, Staphylococcus aureus
YTFTNDIEDK

SEQ ID NO: 26 - protein, 21 aa, Lactobacillus lactis
TVQTNGNQTITSTLNEEQTSK

SEQ ID NO: 27 - protein, 21 aa, Staphylococcus aureus
YKDGIGNYYANLNGSIETFNK

SEQ ID NO: 28 - protein, 19 aa, Staphylococcus aureus
DGIGNYYANLNGSIETFNK

SEQ ID NO: 29 - protein, 13 aa, Staphylococcus aureus
FSHVAFIKPNNGK

SEQ ID NO: 30 - protein, 12 aa, Staphylococcus aureus
TTSVTVTGTLMK

SEQ ID NO: 31 - protein, 13 aa, Lactobacillus lactis
IFEYLGNNEDIAK

SEQ ID NO: 32 - protein, 29 aa, Staphylococcus aureus
FKEVTSNMSGNLNLQNNGSYSLNIENLDK

SEQ ID NO: 33 - protein, 20 aa, Staphylococcus aureus
TYVVHYDGEYLNGTDEVDFR

SEQ ID NO: 34 - protein, 12 aa, Staphylococcus aureus
TQMVGHPEQLYK

SEQ ID NO: 35 - protein, 14 aa, Staphylococcus aureus
EDTIKETLTGQYDK

SEQ ID NO: 36 - protein, 34 aa, Staphylococcus aureus
HHADVVEYEEDTNPGGGQVTTESNLVEFDEESTK

SEQ ID NO: 37 - protein, 17 aa, Lactobacillus lactis
GIVTGAVSDHTTVEDTK

SEQ ID NO: 38 - protein, 31 aa, Lactobacillus lactis
EYTTESNLIELVDELPEEHGQAQGPVEEITK

SEQ ID NO: 39 - protein, 23 aa, Lactobacillus lactis
YEQGGNIVDIDFDSVPQIHGQNK

(19) United States
(12) Patent Application Publication
(10) Pub. No.: US 2015/0265692 A1
(43) Pub. Date: Sep. 24, 2015

(54) IMMUNOGENIC COMPOSITION COMPRISING AN INACTIVATED RECOMBINANT NON-PATHOGENIC BACTERIUM

(71) Applicant: Universite de Lausanne, Lausanne (CH)

(72) Inventors: Jose Entenza, Lausanne (CH); Philippe Moreillon, Lausanne (CH); Tiago Rafael Veloso, Lausanne (CH); Yok-ai Que, Lausanne (CH); Mathilde Ythier, Lausanne (CH)

(21) Appl. No.: 14/426,273

(22) PCT Filed: Sep. 4, 2013
(86) PCT No.: PCT/EP2013/068231

§ 371 Date: Mar. 5, 2015

(30) Foreign Application Priority Data
Sep. 6, 2012 (EP) 12183285.1

(51) Int. Cl. A61K 39/085; C07K 14/31; C07K 16/12

(52) U.S. Cl. CPC A61K 39/085; C07K 16/1271; C07K 14/31; A61K 2039/521

(57) ABSTRACT

The present invention relates to immunogenic compositions, vaccines and antibodies for the treatment and/or prevention of infections and diseases caused by S. aureus in a subject in need thereof.

### FIELD OF THE INVENTION

The present invention relates to immunogenic compositions, vaccines and antibodies for the treatment and/or prevention of infections and diseases caused by S. aureus in a subject in need thereof.

### BACKGROUND OF THE INVENTION

Staphylococcus aureus is a major pathogen responsible for a variety of diseases, from benign skin infections, such as folliculitis and furunculosis, to life-threatening conditions, including erysipelas, deep-seated abscesses, osteomyelitis, pneumonia, sepsis, and infective endocarditis (IE). In addition to infections in which the organism is physically present at the infected site, S. aureus is also capable of producing “distant” diseases, which are mediated by the secretion of toxins. This success is ensured by the coordinated expression of numerous surface adhesins, which mediate host-tissue colonization, and secreted proteins and toxins, which promote invasion as well as strategies to escape the host immune system (Que et al., 2009).

For instance, S. aureus adhesins—which are collectively referred to as MSCRAMMs for Microbial Surface Components Reacting with Adherence Matrix Molecules—encompass at least 21 surface-anchored proteins including fibrinogen-binding proteins A and B (clumping factors A and B, or ClfA and ClfB), fibronectin-binding proteins A and B (FnPBA and FnBPB), collagen-binding protein (Cna) and protein A (Spa) to mention just a few (Patti et al., 1994).

Previous studies have shown that ClfA is essential for the development of IE. Moreover, after individual expression of ClfA in the non-pathogenic bacteria Lactococcus lactis, it has been shown that fibrinogen-binding was pivotal in promoting IE. In addition ClfA also interacts with the GPIIβIIIα receptor on the surface of platelets, a feature that plays an important indirect role in the ability of S. aureus to induce IE (Que et al., 2011).

Numerous attempts to develop vaccines against a variety of these structures, especially against the fibrinogen binding-domain of ClfA, have been attempted with various successes in animal models, but none of them have achieved sustainable efficacy in human clinical trials (Broughan et al., 2011).

In parallel, expressing antigens in non-pathogenic L. lactis has been attempted to trigger mucosal immunity. However, technical issues such as in vivo persistence of the bacterial strain as well as antigen release are as yet incompletely solved (Wells et al., 2008).

However, despite these studies and attempts, there is currently no efficacious immunogenic composition for treating and/or preventing S. aureus infections and diseases caused by S. aureus.

This object has been achieved by providing an immunogenic composition comprising an inactivated recombinant non-pathogenic bacterium, or a part thereof, expressing on its cell surface at least one folded sequence of a S. aureus adhesin, or a sequence having 80% or more sequence identity to said folded sequence of a S. aureus adhesin, wherein said at least one folded sequence of a S. aureus adhesion, or sequence having 80% or more sequence identity to said folded sequence of a S. aureus adhesin, is entirely accessible to trypsin digestion.

### SUMMARY OF THE INVENTION

The invention provides an immunogenic composition comprising an inactivated recombinant non-pathogenic bacterium, or a part thereof, expressing on its cell surface at least one folded sequence of a S. aureus adhesin, or a sequence having 80% or more sequence identity to said folded sequence of a S. aureus adhesin, wherein said at least one folded sequence of a S. aureus adhesion, or sequence having 80% or more sequence identity to said folded sequence of a S. aureus adhesin, is entirely accessible to trypsin digestion.

Furthermore, the invention also provides a vaccine comprising an immunogenic composition of the invention in an immunologically acceptable carrier or diluent.

The invention further provides the use of the vaccine of the invention for the treatment and/or prevention of infections and diseases caused by S. aureus in a subject in need thereof.

Also provided is an isolated and/or purified antibody, antibody fragment or derivative thereof able to bind to the at least one folded sequence of a S. aureus adhesin expressed on the cell surface of an inactivated recombinant non-pathogenic bacterium.

### BRIEF DESCRIPTION OF THE FIGURE

FIG. 1. Prevention of S. aureus experimental endocarditis (i.e. infected vegetations in cardiac valves) in rats immunized with vaccine preparations. Rats were immunized with various vaccine preparations (see infra) for 6 weeks as described, before aortic vegetations were induced. 24 h later, they were challenged with identical inoculum sizes administered either by continuous infusion (0.0017 ml/min over 10 h), or by i.v. bolus (1 ml in 1 min). (A) Animals challenged with S. aureus Newman (10⁴ CFU), and (B) with S. aureus P8 (10⁶ CFU). * P<0.05 compared to the control group by χ² test. Control: group immunized with PBS; Adj.: group immunized with Freund's adjuvant group with the adjuvant emulsified at a 1:1 ratio in PBS; L. lactis plL253, L. lactis ClfA and L. lactis ClfA/FnBPA(CD): groups immunized with L. lactis plL253, ClfA or L. lactis ClfA/FnBPA(CD), respectively, emulsified at a 1:1 ratio in Freund's adjuvant.

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” or “comprising” is generally used in the sense of include/including, that is to say permitting the presence of one or more features or components.

As used in the specification and claims, the singular form “a”, “an” and “the” include plural references unless the context clearly dictates otherwise.

As used herein, “at least one” means “one or more.”

As used herein, the terms “protein”, “polypeptide”, “polypeptidic”, “peptide” and “peptidic” are used interchangeably herein to designate a series of amino acid residues connected to the other by peptide bonds between the alpha-amino and carboxy groups of adjacent residues.

As used herein the term “subject” is well-recognized in the art, and, is used herein to refer to a mammal, including dog, cat, rat, mouse, monkey, cow, horse, goat, sheep, pig, camel, and, most preferably, a human. The term does not denote a particular age or sex. Thus, adult and newborn subjects, whether male or female, are intended to be covered.

The present invention provides an immunogenic composition comprising an inactivated recombinant non-pathogenic bacterium, or a part thereof, expressing on its cell surface at least one folded sequence of a S. aureus adhesin, or a sequence having 80% or more sequence identity to said folded sequence of a S. aureus adhesin, wherein said at least one folded sequence of a S. aureus adhesion, or sequence having 80% or more sequence identity to said folded sequence of a S. aureus adhesin, is entirely accessible to trypsin digestion.

A “non-pathogenic bacterium” refers to a bacterium that does not cause a disease state when in contact with a subject. Examples of non-pathogenic bacteria are selected from the non-limiting group comprising the Bacillus genus, Lactobacillus genus, Lactococcus genus, Sporolactobacillus genus, Bifidobacterium genus, and the like bacteria. Preferably, the non-pathogenic bacterium is selected from the group comprising L. lactis, L. acidophilus and Lactobacilli. Particularly preferred is L. lactis and most particularly preferred is L. lactis subspecies cremoris 1363.

Lactococcus lactis is a nonpathogenic bacterium that is being developed, in its live form, as a vaccine delivery vehicle for immunization by mucosal routes.

However, the non-pathogenic bacterium of the invention is in an inactivated form. Inactivation is performed using methods and/or compounds known in the art, such as for example by H₂O₂, formaldehyde, heat or UV treatments.

The present invention also refers to a part of said inactivated recombinant non-pathogenic bacterium. Usually, this part consists in an isolated and/or purified cell wall of the inactivated recombinant non-pathogenic bacterium.

Generally, the non-pathogenic bacterium of the invention is a recombinant bacterium as it comprises at least one heterologous nucleic acid molecule encoding a folded sequence of a S. aureus adhesin.

Usually, the nucleic acid molecule encoding a folded sequence of a S. aureus adhesin is in the form of deoxyribonucleic acid (DNA). DNA which can be used herein is any polydeoxynuclotide sequence, including, e.g. double-stranded DNA, single-stranded DNA, double-stranded DNA wherein one or both strands are composed of two or more fragments, double-stranded DNA wherein one or both strands have an uninterrupted phosphodiester backbone, DNA containing one or more single-stranded portion(s) and one or more double-stranded portion(s), double-stranded DNA wherein the DNA strands are fully complementary, double-stranded DNA wherein the DNA strands are only partially complementary, circular DNA, covalently-closed DNA, linear DNA, covalently cross-linked DNA, cDNA, chemically-synthesized DNA, semi-synthetic DNA, biosynthetic DNA, naturally-isolated DNA, enzyme-digested DNA, sheared DNA, labeled DNA, such as radiolabeled DNA and fluorochrome-labeled DNA, DNA containing one or more non-naturally occurring species of nucleic acid, genomic or complementary DNA. Preferably, the DNA is a genomic DNA or a complementary DNA (cDNA).

DNA sequences that encode a folded sequence of a S. aureus adhesin can be synthesized by standard chemical techniques, for example, the phosphotriester method or via automated synthesis methods and PCR methods.

The DNA sequence encoding a folded sequence of a S. aureus adhesin according to the invention may also be produced by enzymatic techniques. Thus, restriction enzymes, which cleave nucleic acid molecules at predefined recognition sequences can be used to isolate nucleic acid sequences from larger nucleic acid molecules containing the nucleic acid sequence, such as DNA (or RNA) that codes for a peptide consisting a folded sequence of a S. aureus adhesin.

Encompassed by the present invention is also a nucleic acid in the form of a polyribonucleotide (RNA), including, e.g., single-stranded RNA, cRNA, double-stranded RNA, double-stranded RNA wherein one or both strands are composed of two or more fragments, double-stranded RNA wherein one or both strands have an uninterrupted phosphodiester backbone, RNA containing one or more single-stranded portion(s) and one or more double-stranded portion(s), double-stranded RNA wherein the RNA strands are fully complementary, double-stranded RNA wherein the RNA strands are only partially complementary, covalently crosslinked RNA, enzyme-digested RNA, sheared RNA, mRNA, chemically-synthesized RNA, semi-synthetic RNA, biosynthetic RNA, naturally-isolated RNA, labeled RNA, such as radiolabeled RNA and fluorochrome-labeled RNA, RNA containing one or more non-naturally-occurring species of nucleic acid.

The present invention also includes variants of the aforementioned sequences that are nucleotide sequences that vary from the reference sequence by conservative nucleotide substitutions, whereby one or more nucleotides are substituted by another with same characteristics.

The invention also encompasses allelic and polymorphic variants of the aforementioned sequences; that is, naturally-occurring alternative forms of the folded sequence of a S. aureus adhesin that also encode peptides that are identical, homologous or related to that encoded by the sequence of a S. aureus adhesin. Alternatively, non-naturally occurring variants may be produced by mutagenesis techniques or by direct synthesis.

Also encompassed in the present invention is a sequence having 80% or more sequence identity to said folded sequence of a S. aureus adhesin. The percentage identity of a polynucleotide or polypeptide sequence is determined by aligning polynucleotide and polypeptide sequences; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity. Preferably, the sequence has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to said folded sequence of a S. aureus adhesin.

In case the sequence of a S. aureus adhesin is the clumping factor A (ClfA) then the acid nucleic molecule encoding the folded sequence of a S. aureus adhesin, namely the ClfA, is a genomic DNA as set forth in SEQ ID No 2.

TABLE 1
SEQ ID No 2

| | |
|---|---|
| 1 | ggtaccataa attacacatc tgcttttgaa aaaatatgat |
| | ttcaagctag gattacatta |
| 61 | ggtagagttc atattaataa taaaaaatgt ttgcaatcaa |
| | atcgtacgtt gtcgtttgta |
| 121 | attcttaaaa tagcaataaa taaaatgttt gttagtaaag |
| | tattattgtg gataataaaa |
| 181 | tatcgataca aattaattgc tataatgcaa ttttagtgta |
| | taattccatt aacagagatt |
| 241 | aaatatatct ttaaagggta tatagttaat ataaaatgac |
| | tttttaaaaa gagggaataa |
| 301 | aatgaatatg aagaaaaaag aaaaacacgc aattcggaaa |
| | aaatcgattg gcgtggcttc |
| 361 | agtgcttgta ggtacgttaa tcggttttgg actactcagc |
| | agtaaagaag cagatgcaag |
| 421 | tgaaaatagt gttacgcaat ctgatagcgc aagtaacgaa |
| | agcaaaagta atgattcaag |
| 481 | tagcgttagt gctgcaccta aaacagacga cacaaacgtg |
| | agtgatacta aaacatcgtc |
| 541 | aaacactaat aatggcgaaa cgagtgtggc gcaaaatcca |
| | gcacaacagg aaacgacaca |
| 601 | atcatcatca acaaatgcaa ctacggaaga aacgccggta |
| | actggtgaag ctactactac |
| 661 | gacaacgaat caagctaata caccggcaac aactcaatca |
| | agcaatacaa atgcggagga |
| 721 | attagtgaat caaacaagta atgaaacgac ttttaatgat |
| | actaatacag tatcatctgt |
| 781 | aaattcacct caaaattcta caaatgcgga aaatgtttca |
| | acaacgcaag atacttcaac |
| 841 | tgaagcaaca ccttcaaaca atgaatcagc tccacagagt |
| | acagatgcaa gtaataaaga |
| 901 | tgtagttaat caagcggtta atacaagtgc gcctagaatg |
| | agagcattta gtttagcggc |
| 961 | agtagctgca gatgcaccgg cagctggcac agatattacg |
| | aatcagttga cgaatgtgac |
| 1021 | agttggtatt gactctggta cgactgtgta tccgcaccaa |
| | gcaggttatg tcaaactgaa |
| 1081 | ttatggtttt tcagtgccta attctgctgt taaaggtgac |
| | acattcaaaa taactgtacc |
| 1141 | taaagaatta aacttaaatg gtgtaacttc aactgctaaa |
| | gtgccaccaa ttatggctgg |
| 1201 | agatcaagta ttggcaaatg gtgtaatcga tagtgatggt |
| | aatgttattt atacatttac |
| 1261 | agactatgta aatactaaag atgatgtaaa agcaactttg |
| | accatgcccg cttatattga |
| 1321 | ccctgaaaat gttaaaaaga caggtaatgt gacattggct |
| | actggcatag gtagtacaac |
| 1381 | agcaaacaaa acagtattag tagattatga aaaatatggt |
| | aagttttata acttatctat |
| 1441 | taaaggtaca attgaccaaa tcgataaaac aaataatacg |
| | tatcgtcaga caatttatgt |
| 1501 | caatccaagt ggagataacg ttattgcgcc ggttttaaca |
| | ggtaatttaa aaccaaatac |
| 1561 | ggatagtaat gcattaatag atcagcaaaa tacaagtatt |
| | aaagtatata aagtagataa |
| 1621 | tgcagctgat ttatctgaaa gttactttgt gaatccagaa |
| | aactttgagg atgtcactaa |
| 1681 | tagtgtgaat attacattcc caaatccaaa tcaatataaa |
| | gtagagttta atacgcctga |
| 1741 | tgatcaaatt acaacaccgt atatagtagt tgttaatggt |
| | catattgatc cgaatagcaa |
| 1801 | aggtgattta gctttacgtt caactttata tgggtataac |
| | tcgaatataa tttggcgctc |
| 1861 | tatgtcatgg gacaacgaag tagcatttaa taacggatca |
| | ggttctggtg acggtatcga |
| 1921 | taaaccagtt gttcctgaac aacctgatga gcctggtgaa |
| | attgaaccaa ttccagagga |
| 1981 | ttcagattct gacccaggtt cagattctgg cagcgattct |
| | aattcagata gcggttcaga |
| 2041 | ttcgggtagt gattctacat cagatagtgg ttcagattca |
| | gcgagtgatt cagattcagc |
| 2101 | aagtgattca gactcagcga gtgattcaga ttcagcaagc |
| | gattccgact cagcgagcga |
| 2161 | ttccgactca gacaatgact cggattcaga tagcgattct |
| | gactcagaca gtgactcaga |
| 2221 | ttccgacagt gactcagatt cagatagcga ttctgactca |
| | gacagtgact cagattcaga |
| 2281 | tagcgattca gattcagata gcgattcaga ttccgacagt |
| | gattccgact cagacagcga |
| 2341 | ttctgactcc gacagtgatt ccgactcaga cagcgattca |
| | gattccgaca gtgattccga |
| 2401 | ctcagatagc gattccgact cagatagcga ctcagattca |
| | gacagcgatt cagattcaga |
| 2461 | cagcgattca gattcagata gcgattcaga ttccgacagt |
| | gactcagatt ccgacagtga |
| 2521 | ctcggattca gatagcgatt cagattccga cagtgactca |
| | gattccgaca gtgactcaga |
| 2581 | ctcagacagt gattcggatt cagcgagtga ttcggattca |
| | gatagtgatt ccgactccga |
| 2641 | cagtgactcg gattcagata gcgactcaga ctcggatagc |
| | gactcggatt cagatagcga |
| 2701 | ttcggactca gatagcgatt cagaatcaga cagcgattca |
| | gaatcagaca gcgattcaga |
| 2761 | ttcagacagc gactcagaca gtgactcaga ttcagatagt |
| | gactcggatt cagcgagtga |
| 2821 | ttcagactca ggtagtgact ccgattcatc aagtgattcc |
| | gactcagaaa gtgattcaaa |
| 2881 | tagcgattcc gagtcaggtt ctaacaataa tgtagttccg |
| | cctaattcac ctaaaaatgg |
| 2941 | tactaatgct tctaataaaa atgaggctaa agatagtaaa |
| | gaaccattac cagatacagg |
| 3001 | ttctgaagat gaagcaaata cgtcactaat ttggggatta |
| | ttagcatcaa taggttcatt |
| 3061 | actacttttc agaagaaaaa aagaaaataa agataagaaa |
| | taagtaataa tgatattaaa |
| 3121 | ttaatcatat gattcatgaa gaagccacct taaaaggtgc |
| | ttcttttact tggattttcc |
| 3181 | aaatatattg tttgaatata attaataatt aattcatcaa |
| | cagttaatta ttttaaaaag |
| 3241 | gtagatgtta tataatttgg cttggcgaaa aaatagggtg |
| | taaggtaggt tgttaattag |
| 3301 | ggaaaattaa ggagaaaata cagttgaaaa ataaattgct |
| | agttttatca ttgggagcat |
| 3361 | tatgtgtatc acaaatttgg gaaagtaatc gtgcgagtgc |
| | agtggtttct ggggagaaga |
| 3421 | atccatatgt atctgagtcg ttgaaactga ctaataataa |
| | aaataaatct agaacagtag |
| 3481 | aagagtataa gaaaagctt |

In case the sequence of a S. aureus adhesin is the fibronectin-binding protein A (FnPBA) then the acid nucleic molecule encoding the folded sequence of a S. aureus adhesin, namely the FnPBA, is preferably a genomic DNA as set forth in SEQ ID No 3.

TABLE 2
SEQ ID No 3

| | |
|---|---|
| 1 | ttatgctttg tgattctttt tatttctgcg taataatgct |
| | aaacctagaa tgctgaataa |
| 61 | tccgccgaac aacatacctt tgtttgttga ttcttctcca |
| | cctgtttcag gtagttcaga |
| 121 | tttcttagat tgtggttttt tagttggtgc cactgcttta |
| | accttttcat tgatttcaat |
| 181 | aacaggtgtt actactttac cttgttccac tggtttagaa |
| | ggctttttag gttcttcttt |
| 241 | ggcaggtggt actggtttac caggttcagc tggtacctct |
| | ggtgttggcg gtgttggagt |
| 301 | ttctggctca ctcggcactt ctggtgtcgg tggtgttggt |
| | gtttccggct cacttggtac |
| 361 | ttctggtgtt ggtggcgttg gtgtttccgg ctcacttggt |
| | acttctggtg tcggtggcgt |
| 421 | tggtggcacg attggaggtg ttgtatcttc ttcaatcgtt |
| | tgttgacctt cattttggcc |
| 481 | gcttactttt ggaagtgtat cttcttcaaa gtcaacacta |
| | ttgtgtccac cgaattgata |
| 541 | acttggttta tctttatttg tatcttcttc aataatttca |
| | gtgtgcttat tgaatccgtg |
| 601 | aatatgtggc acactgtcga agtcgatatc aatgatgtta |
| | ccgccatgtt catacttagg |
| 661 | tttgtctttt tctgtatctt cctcgaatga ctgattacct |
| | ttattttgac catgaatttg |
| 721 | aggtacacta tcaaaatcga tatctacgat attgccacct |
| | tgttcatatt taggtttgtc |
| 781 | ttcttctgtg tcttcctcga atgactggtt accgctattt |
| | tggccacctt cataacctaa |
| 841 | ttcactctta atatcaacgt ggctattttc ttcgatttct |
| | tcaatcacgt cataattccc |
| 901 | gtgaccattt tcagttccta aaccagaatg agaaatatga |
| | tgattgtttt tagtaatttc |
| 961 | ctcgactggt ccttgtgctt gaccatgctc ttcaggtaat |
| | tcatccacta attcaatcag |
| 1021 | attactttca gttgtatatt ctttcgtatc ttcaactgtt |
| | gtatgatcgc tcactgcgcc |
| 1081 | agttacaata ccttttgtag actcttcgtc aaattcaact |
| | aagttagact cagtagtaac |
| 1141 | ctgaccacca cctgggtttg tatcttcttc atattcaaca |
| | acatcagcgt gatgttttga |
| 1201 | attttcatgt gtagattctt caaagtcaat tggatttgat |
| | tcctcagagg actcagtgta |
| 1261 | tcctccaacg tgacctgctt cgctatccac agcagtatgg |
| | taatcgatat caatagctga |
| 1321 | tgaatccgtt tcttctattg tttcaatgta tccatcaaca |
| | tatccacctc caccatctat |
| 1381 | agctgtgtgg taatcaatgt caagagttga tgaatcatat |
| | tcctcttcaa cagtagttac |
| 1441 | taaattctta tcatattgac ctgtaagagt ttctttaatt |
| | gtatcttctt tatattcaaa |
| 1501 | tttattattt tgaataatcg gaccattttt ctcatttccg |
| | ttcgctttat tactgtataa |
| 1561 | aactaaacca ttatcccaag ttaaggtata tcctctatca |
| | taataatact tataaagttg |
| 1621 | ctctggatgt cctaccattt gtgttctaaa atcaacttca |
| | tcagtaccat ttaaatactc |
| 1681 | tccatcatag tgaacaacat aagttttatc tagattttct |
| | atattcaatg aatagcttcc |
| 1741 | attattttgt aaattcaaat tcccactcat attacttgtg |
| | acttctttaa atttagaagt |
| 1801 | atctgtcgta tttgcatata cactcttcgc tatgtcttca |
| | ttattaccca agtattcaaa |
| 1861 | tatcctaact tttggttgat ttccattctg attactacct |
| | ttcattaaag ttccagtaac |
| 1921 | agtcacactt gtcgttttac cattattagg tttaataaat |
| | gcaacatgcg aaaatctatt |
| 1981 | attcgcttta ttaaatgtct caatcgatcc atttaaattg |
| | gcataataat tcccaatacc |
| 2041 | atctttatat ttaacatcta attcctttga agtttgttct |
| | tcatttagtg ttgaagttat |
| 2101 | agtttgattt ccattagttt gtacagtttt aggatcaata |
| | aataaattaa tttctagttc |
| 2161 | agccgttaca tcaaccttat cttcaatatc atttgtaaat |
| | gtatatctaa tctttccacc |
| 2221 | ttctaaaact tcacctgtcg ccattacgac tgaaccattt |
| | ttaatttctg gtacttttct |
| 2281 | agcagttgat acgccatgcg tatttacatt atttgataaa |
| | gtaaagtcaa agtagtcacc |
| 2341 | ttgatgtaaa ccattctcaa atttcaactt atattttagt |
| | accgctcgtt gtcctgcatg |
| 2401 | aggttctact ttatttgtat tgttatgccc ctcaatagaa |
| | ccaatttcta ctgtaacttt |
| 2461 | acttgttaca tctgtacccg tttccacttt cgcgttacta |
| | gcttccttag cttccgctac |
| 2521 | atctgctgat cttgtcacac gtggcttact ttctgatgcc |
| | gttcttggct gtgccacttc |
| 2581 | aacttgtgtt tctgcgactt gattttgtgt agccttttta |
| | ggtgttaaat ctacttgtct |
| 2641 | ttgatctccg ctattgtctt gagattgtgt tgtttcctta |
| | acttgaggtt tcgcttcttc |
| 2701 | cttaactacc tcttctttaa ctgtttctat atttgctggt |
| | tgtgcagttt gtggtgcttg |
| 2761 | tactgctttt ggtgcttctt cagttgttac ttgtgttgcg |
| | tttgacggtt gttctgttac |
| 2821 | tgttgcgtta tatgattgag tttcttctat atgattaacg |
| | ttagttgcag ttgtttgtgt |
| 2881 | ttcacttgtt ttattatcag tagctgaatt cccattttct |
| | tctactgtag ttgtcttttg |
| 2941 | ttctgatgct gcagcttctt tgtcttgtcc cat |

Previously, it has been shown that S. aureus proteins expressed on the surface of Lactococci were entirely accessible to trypsin digestion for analysis by LC-MS (liquid chromatography coupled with mass spectrometry) (Ythier et al., 2012). In contrast, this was not the case in S. aureus, where trypsin had a limited access to the surface proteins, indicating that part of their structure was embedded in the wall and inaccessible to digestion, and thus to recognition by antibodies as well. The trypsin shaving protocol is a classical protocol described in Ythier et al. and in the Example parts.

The trypsin digestion of adhesins such as, for example, ClfA expressed on the surface of lactococci and/or FnPBA expressed on the surface of Lactococci, releases a digestion profile which is different from the digestion profile of the same adhesins expressed on the surface of S. aureus.

For example, the digestion profile of ClfA expressed on the surface of lactococci reveals 10 peptides versus 9 for ClfA expressed on the surface of S. aureus. Among these 10 peptides, 3 peptides (SEQ IDs 5, 6, and 12) were specific of Lactococci.

TABLE 3
Peptides released after trysin digestion of ClfA expressed on

| SEQ | | |
|---|---|---|
| ID/Position | | |
| (aa) | S. aureus | L. lactis |
| SEQ ID No 5 | | SENSVTQSDSASNESK |
| 40-55 | | |
| SEQ ID No 6 | | SNDSSSVSAAPK |
| 56-67 | | |
| SEQ ID No 7 | DVVNQAVNTSAPR | DVVNQAVNTSAPR |
| 200-212 | | |
| SEQ ID No 8 | LNYGFSVPNSAVK | LNYGFSVPNSAVK |
| 259-271 | | |
| SEQ ID No 9 | ELNLNGVTSTAK | |
| 282-293 | | |
| SEQ ID No 10 | ATLTMPAYIDPENVK | ATLTMPAYIDPENVK |
| 331-345 | | |
| SEQ ID No 11 | TGNVTLATGIGSTTANK | TGNVTLATGIGSTTANK |
| 347-363 | | |
| SEQ ID No 12 | | FYNLSIK |
| 375-381 | | |
| SEQ ID No 13 | QTIYVNPSGDNVIAPVLTGNLKPNTD | QTIYVNPSGDNVIAPVLTGNLKPNTDSN |
| 396-434 | | ALIDQQNTSISNALIDQQNTSI |
| | K | K |
| SEQ ID No 14 | VDNAADLSESYFVNPENFEDVTNSVN | |
| 438-473 | ITFPNPNQYK | |
| SEQ ID No 15 | VEFNTPDDQITTPYIVVVNGHIDPNSK | VEFNTPDDQITTPYIVVVNGHIDPNSK |
| 474-500 | | |

As another example, the digestion profile of FnbpA expressed on the surface of Lactococci reveals 10 peptides versus 22 for FnbpA expressed on the surface of S. aureus. Among these peptides, 2 peptides (SEQ IDs 38 and 39) were specific of lactococci.

TABLE 4
Peptides released after trysin digestion of FnbpA expressed on

| Position | | |
|---|---|---|
| (aa) | S. aureus | L. lactis |
| SEQ ID No 16 | TSETQTTATNVNHIEETQSYNATVTEQPSNATQ | TSETQTTATNVNHIEETQSYNAT |
| 57-97 | VTTEEAPK | VTEQPSNATQVTTEEAPK |
| SEQ ID No 17 | AVQAPQTAQPANIETVK | AVQAPQTAQPANIETVK |
| 98-114 | | |
| SEQ ID No 18 | AVQAPQTAQPANIETVKEEVVK | |
| 98-119 | | |
| SEQ ID No 19 | EEVVKEEAKPQVK | |
| 115-127 | | |
| SEQ ID No 20 | KATQNQVAETQVEVAQPR | KATQNQVAETQVEVAQPR |
| 148-165 | | |
| SEQ ID No 21 | ATQNQVAETQVEVAQPR | ATQNQVAETQVEVAQPR |
| 149-165 | | |
| SEQ ID No 22 | VTVEIGSIEGHNNTNK | VTVEIGSIEGHNNTNK |
| 201-216 | | |
| SEQ ID No 23 | FENGLHQGDYFDFTLSNNVNTHGVSTAR | |
| 233-260 | | |
| SEQ ID No 24 | NGSWMATGEVLEGGK | |
| 267-282 | | |
| SEQ ID No 25 | YTFTNDIEDK | |
| 285-294 | | |
| SEQ ID No 26 | TVQTNGNQTITSTLNEEQTSK | TVQTNGNQTITSTLNEEQTSK |
| 311-331 | | |
| SEQ ID No 27 | YKDGIGNYYANLNGSIETFNK | |
| 337-357 | | |
| SEQ ID No 28 | DGIGNYYANLNGSIETFNK | |
| 339-357 | | |
| SEQ ID No 29 | FSHVAFIKPNNGK | |
| 362-374 | | |
| SEQ ID No 30 | TTSVTVTGTLMK | |
| 375-386 | | |
| SEQ ID No 31 | IFEYLGNNEDIAK | IFEYLGNNEDIAK |
| 399-411 | | |
| SEQ ID No 32 | FKEVTSNMSGNLNLQNNGSYSLNIENLDK | |
| 423-451 | | |
| SEQ ID. 33 | TYWHYDGEYLNGTDEVDFR | |
| 452-471 | | |
| SEQ ID No 34 | TQMVGHPEQLYK | |
| 472-483 | | |
| SEQ ID No 35 | EDTIKETLTGQYDK | |
| 524-537 | | |
| SEQ ID No 36 | HHADVVEYEEDTNPGGGQVTTESNLVEFDEESTK | |
| 622-655 | | |
| SEQ ID No 37 | GIVTGAVSDHTTVEDTK | GIVTGAVSDHTTVEDTK |
| 656-672 | | |
| SEQ ID No 38 | | EYTTESNLIELVDELPEEHGQAQ |
| 673-702 | | GPVEEITK |
| SEQ ID No 39 | | YEQGGNIVDIDFDSVPQIHGQNK |
| 764-786 | | |

Since all these domains are variously exposed on the S. aureus surface, the present invention shows that vaccination against only one binding-domain, which might become hidden in certain circumstances, is less effective than vaccination against the whole protein presented in a functional conformation on the surface of Lactococci.

Preferably, the sequence of the S. aureus adhesin of the invention is a folded sequence.

A “folded sequence” refers to a protein that is structured under physiologic conditions. Alternatively, the sequence of the invention is a sequence having 80% or more sequence identity to said folded sequence of a S. aureus adhesin. Preferably, the S. aureus adhesin is selected from the group comprising fibrinogen-binding protein A (clumping factor A; ClfA), fibrinogen-binding protein B (ClfB), fibronectin-binding protein

A (FnPBA) and fibronectin-binding protein B (FnBPB), collagen-binding protein (Cna) and protein A (Spa), Serine-aspartate repeat protein C, D and E (SdrC-E), Plasmin-sensitive protein (PIs), Factor affecting methicillin resistance in the presence of Triton X-100 (FmtB), surface protein A-K (SasA-K). A combination of one or more of the above listed S. aureus adhesin sequences is also envisioned.

Most preferably, the S. aureus adhesin is the fibrinogen-binding protein A (clumping factor A; ClfA). Clumping factor A (ClfA) is an MSCRAMM protein expressed by S. aureus that promotes binding of fibrinogen and fibrin to the bacterial cell surface. ClfA is the prototype of a recently identified multigene family of cell surface proteins characterized by a common domain composed of a unique serine-aspartate repeat. The gene encoding the fibrinogen-binding protein shows a 933-amino-acid polypeptide that contains structural features characteristic of many cell surface-associated proteins from gram-positive bacteria, including a typical cell wall attachment region comprising an LPXTG motif, a hydrophobic transmembrane sequence, and a positively charged C terminus. The fibrinogen-binding domain of ClfA has been localized to a 218-residue segment within region A.

More preferably, the sequence of the invention consists in a whole functional amino acid sequence of ClfA (amino acid 1 to 933, table 5).

TABLE 5
SEQ ID No 1
MNMKKKEKHAIRKKSIGVASVLVGTLIGFGLLSSKEADASENSVTQSDS
ASNESKSNDSSSVSAAPKTDDTNVSDTKTSSNTNNGETSVAQNPAQQET
TQSSSTNATTEETPVTGEATTTTTNQANTPATTQSSNTNAEELVNQTSN
ETTFNDTNTVSSVNSPQNSTNAENVSTTQDTSTEATPSNNESAPQSTDA
SNKDVVNQAVNTSAPRMRAFSLAAVAADAPAAGTDITNQLTNVTVGIDS
GTTVYPHQAGYVKLNYGFSVPNSAVKGDTFKITVPKELNLNGVTSTAKV
PPIMAGDQVLANGVIDSDGNVIYTFTDYVNTKDDVKATLTMPAYIDPEN
VKKTGNVTLATGIGSTTANKTVLVDYEKYGKFYNLSIKGTIDQIDKTNN
TYRQTIYVNPSGDNVIAPVLTGNLKPNTDSNALIDQQNTSIKVYKVDNA
ADLSESYFVNPENFEDVTNSVNITFPNPNQYKVEFNTPDDQITTPYIVV
VNGHIDPNSKGDLALRSTLYGYNSNIIWRSMSWDNEVAFNNGSGSGDGI
DKPVVPEQPDEPGEIEPIPEDSDSDPGSDSGSDSNSDSGSDSGSDSTSD
SGSDSASDSDSASDSDSASDSDSASDSDSASDSDSDNDSDSDSDSDSDS
DSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDS
DSDSDSDSDSDSDSDSDSDSDSDSDSDSDSDSASDSDSDSDSDSDSDSD
SDSDSDSDSDSDSDSDSDSDSDSESDSDSESDSDSDSDSDSDSDSDSDS
DSDSASDSDSGSDSDSSSDSDSESDSNSDSESGSNNNVVPPNSPKNGTN
ASNKNEAKDSKEPLPDTGSEDEANTSLIWGLLASIGSLLLFRRKKENKD
KK

More preferably also, the sequence of the invention consists in a whole functional amino acid sequence of FnPBA (amino acid 1 to 990, table 6).

TABLE 6
SEQ ID No 4
MGQDKEAAASEQKTTTVEENGNSATDNKTSETQTTATNVNHIEETQSY
NATVTEQPSNATQVTTEEAPKAVQAPQTAQPANIETVKEEVVKEEAKP
QVKETTQSQDNSGDQRQVDLTPKKATQNQVAETQVEVAQPRTASESKP
RVTRSADVAEAKEASNAKVETGTDVISKVTVEIGSIEGHNNTNKVEPH
AGQRAVLKYKLKFENGLHQGDYFDFTLSNNVNTHGVSTARKVPEIKNG
SVVMATGEVLEGGKIRYTFTNDIEDKVDVTAELEINLFIDPKTVQTNG
NQTITSTLNEEQTSKELDVKYKDGIGNYYANLNGSIETFNKANNRFSH
VAFIKPNNGKTTSVTVTGTLMKGSNQNGNQPKVRIFEYLGNNEDIAKS
VYANTTDTSKFKEVTSNMSGNLNLQNNGSYSLNIENLDKTYVVHYDGE
YLNGTDEVDFRTQMVGHPEQLYKYYYDRGYTLTWDNGLVLYSNKANGN
EKNGPIIQNNKFEYKEDTIKETLTGQYDKNLVTTVEEEYDSSTLDIDY
HTAIDGGGGYVDGYIETIEETDSSAIDIDYHTAVDSEAGHVGGYTESS
EESNPIDFEESTHENSKHHADVVEYEEDTNPGGGQVTTESNLVEFDEE
STKGIVTGAVSDHTTVEDTKEYTTESNLIELVDELPEEHGQAQGPVEE
ITKNNHHISHSGLGTENGHGNYDVIEEIEENSHVDIKSELGYEGGQNS
GNQSFEEDTEEDKPKYEQGGNIVDIDFDSVPQIHGQNKGNQSFEEDTE
KDKPKYEHGGNIIDIDFDSVPHIHGFNKHTEIIEEDTNKDKPSYQFGG
HNSVDFEEDTLPKVSGQNEGQQTIEEDTTPPIVPPTPPTPEVPSEPET
PTPPTPEVPSEPETPTPPTPEVPSEPETPTPPTPEVPAEPGKPVPPAK
EEPKKPSKPVEQGKVVTPVIEINEKVKAVAPTKKPQSKKSELPETGGE
ESTNKGMLFGGLFSILGLALLRRNKKNHKA

Surprisingly, the Applicants of the present invention have successfully prevented S. aureus experimental endocarditis in rats vaccinated with UV-killed L. lactis expressing heterologously the staphylococcal adhesin ClfA, or ClfA/FnBPA (CD). The success of vaccination is due to the method of antigen delivery, i.e. a whole functional S. aureus surface adhesin on a genuine bacterial surface, rather than only restricted peptides of the protein. This mode of delivery largely increases the repertoire of anti-staphylococcal antibodies generated by the host, and thus increases the efficacy of protection.

The cell wall anchored fibrinogen-binding protein ClfA has been the major target of vaccine candidates, due to its ability to binds to the γ-chain of the fibrinogen. Indeed, previous studies have shown that ClfA is essential for the development of IE due to the pivotal role of fibrinogen-binding (Moreillon et al., 1995; Yok-ai Que et al., 2005). However, numerous attempts to develop vaccines against a variety of S. aureus virulence factors have been attempted with various successes in animal models, but have as yet not achieved sustainable efficacy in human clinical trials.

The reasons for these failures are not entirely clarified, but there are at least two parameters of the S. aureus camouflage system that might have been underestimated until now. First, the fact that the bacterium may vary the way it exposes its antigenic structure on the surface, and second the fact that different strains may undergo antigenic variations. As a result, vaccination against one particular structure that is valid against one strain may not be efficacious against another strain. Therefore, a blocking or opsonizing antibody that is active in certain circumstances may become inactive in other settings.

Staphylococcal adhesins of the LPXTG-protein family are equipped with a spacer domain between the cell wall anchor and the outermost binding domain. This spacer is important to expose the binding domain on top of the plethora of other constituents of the staphylococcal envelope, in order to bind to the target host tissue. Thus, depending on the length of this spacer (which may vary between strains) and the size of other surface components (for instance polysaccharides and protein A), the binding domain of the adhesin may become embedded in other surface structures and hidden to the immune system (Scarpa et al., 2010). Indeed, it was shown that S. aureus exposed differently the fibrinogen-binding protein domain “A” of ClfA (Mcdevitt et al., 1994), as well as the surface capsule, at various stages of in vivo infection (Risley et al., 2007).

Another example comes from the length of anti-phagocytic protein A, which binds antibodies by their Fc fragment. It was recently shown that a longer spacer sequence allowed protein A to better prevent binding of antibodies to various antigenic structures presented on the staphylococcal surface (Scarpa et al., 2010). Moreover, the polymorphism of the protein A gene may affect the efficacy of vaccines in a strain-dependent manner. Indeed, the length of the spacer is determined by series of repeats that are known to vary between different staphylococcal strains, and thus are currently used as phylogenic markers (Kuhn et al., 2007).

Eventually, S. aureus produces a plethora of toxins and superantigens that may interfere with the immune response, and thus help the organism to circumvent existing host immune strategies. Vaccination against such structures were also recently attempted (Broughan et al., 2011).

The importance of antigenic variation in S. aureus is less clear. Indeed, only two major capsular types (types 5 and 8) are implicated in infection in human, and surface proteins and toxins are relatively well conserved (Shinefield et al., 2002). However, many gram-positive pathogens, including group A streptococci and Streptococcus pneumoniae, undergo wide genetic variability at the level of the anti-phagocytic M protein and the polysaccharidic capsule, respectively. Taken together, the current consensus for anti-S. aureus vaccines is that they should comprise several antigens in order to be effective, and that single antigen-based vaccines are bound to fail (Broughan et al., 2011). However, the ideal antigen mixture to be used has yet to be elucidated.

Examples showed hereafter that S. aureus adhesins could be expressed functionally in L. Lactis in vitro, and could promote experimental endocarditis by the recombinant Lactococci in vivo. When conjugated with the proteomic dissection of the surface proteome of S. aureus, it appeared that the S. aureus proteins expressed on the surface of Lactococci were entirely accessible to trypsin digestion for analysis by LC-MS (liquid chromatography coupled with mass spectrometry) (Ythier et al., 2012). In contrast, this was not the case in S. aureus, where trypsin had a limited access to the surface proteins, indicating that part of their structure was embedded in the wall and inaccessible to digestion, and thus to recognition by antibodies as well. Since all these domains were variously exposed on the S. aureus surface, it was conceivable that vaccination against only one binding-domain, which might become hidden in certain circumstances, might be less effective than vaccination against the whole protein presented in a functional conformation on the surface of lactococci.

As shown in the examples, immunizing series of rats with UV-killed lactococci expressing S. aureus ClfA, or ClfA/FnBPA (CD) protected animals from subsequent experimental endocarditis due to S. aureus induced by low-grade bacteremia (P<0.05 when compared to the several control groups). Remarkably, when the same vaccination schedule was tested in animals where the experimental endocarditis due to S. aureus was induced by high-grade bacteremia (traditional bolus infection) the infection rates increased (2/15 vs 6/6) and the protective effect was lost.

Attempts to vaccinate against severe infections in animal models are usually biased by the fact that most protocols administer very large bacterial inocula, in order to ensure that all untreated control animals will become infected. However, such large inocula—often in the range of 10 s of million bacteria injected intravenously—are incommensurably larger than inoculum sized expected during “natural” infection in human. Thus, it is possible that such large inocula may overwhelm the immune system, and falsely underestimate the efficacy of preventive or therapeutic strategies that would otherwise be efficacious. The Applicants recently showed that this was realistic in the model of experimental endocarditis, and that challenging animals with continuous low-grade bacteremia was as infectious that transient high-grade inoculation, but represented much more the reality of the disease in human (Veloso et al., 2011). Importantly, the good results of the vaccination strategy reported above were achieved with this very realistic model. Indeed, vaccination was less effective against the standard high-grade bacteremia model, an observation that could also explain failures with other types of potential anti-S. aureus vaccines tested in animals in the past, and abandoned.

The present invention also concerns a vaccine. Preferably, the vaccine comprises an immunogenic composition of the invention in an immunologically acceptable carrier or diluent. Preferably said vaccine is for treating and/or preventing infections and diseases caused by S. aureus. Examples of diseases caused by S. aureus are folliculitis, furunculosis, erysipelas, deep-seated abscesses, osteomyelitis, pneumonia, sepsis, and infective endocarditis (IE).

The vaccine of the invention may also contain several antibodies or antibody fragments, e.g. two or three antibodies or antibody fragments that recognize(s) at least one folded sequence of a S. aureus adhesin expressed on the cell surface of an inactivated recombinant non-pathogenic bacterium of the invention.

The immunologically acceptable carrier is usually selected from the group comprising polysaccharide materials forming hydrogels, bacterial ghosts and vesicular carriers. Preferably, the vesicular carriers are selected from the group comprising liposomes, niosomes, transfersomes, and ethosomes, and others known in the art.

Hydrogels envisioned as immunologically acceptable carrier as know in the art and are particularly adapted for mucosal, topical, oral or injectable delivery.

The vaccines of the invention may further comprise one or more adjuvant. Adjuvants can include, but are not limited to, MPL+TDM+CWS (SIGMA), MF59 (an oil-in-water emulsion that includes 5% squalene, 0.5% sorbitan monoleate and 0.5% sorbitan trioleate Chiron), Heat-labile toxin (HLT), CRMig (nontoxic genetic mutant of diphtheria toxin), Squalene (IDEC PHARMACEUTICALS CORP.), Ovalbumin (SIGMA), Quil A (SARGEANT, INC.), Aluminum phosphate gel (SUPERFOS BIOSECTOR), Cholera holotoxin (CT LIST BIOLOGICAL LAB.), Cholera toxin B subunit (CTB), Cholera toxin A subunit-Protein A D-fragment fusion protein, Muramyl dipeptide (MDP), Adjumera (polyphosphazene, VIRUS RESEARCH INSTITUTE), Montanide ISA 720, SPT (an emulsion of 5% squalene, 0.2% Tween 80, 1. 25% Pluronic L121 with phosphate-buffered saline ph 7. 4), Avridine (M6 PHARMACEUTICALS), Bay R1005 (BAYER), Calcitrol (SIGMA), Calcium phosphate gel (SARGEANT INC.), CRL 1005 (Block co-polymer P1205, VAXCEL CORP.), DHEA (MERCK), DMPC (GENZYME PHARMACEUTICALS and FINE CHEMICALS). DMPG (GENZYME PHARMACEUTICALS and FINE CHEMICALS), Gamma Inulin, Gerbu Adjuvant (CC BIOTECH CORP.), GM-CSF, (IMMUNE CORP.), GMDP (PEPTECH LIMITED), Imiquimod (3M PHARMACEUTICALS), ImmTher (ENDOREX CORPORATION), ISCOMTM (ISCOTEC AB), Iscoprep 7.0. 3 TM (ISCOTEC AB), Loxoribine, LT-Oral Adjuvant (E. coli labile enterotoxin, protoxin, BERNA PRODUCTS CORP.), MTP-PE (CIBA-GEIGY LTD), Murametide, (VACSYN S. A.), Murapalmitine (VACSYN S. A.), Pluronic L121 (IDEC PHARMACEUTICALS CORP.), PMMA (INSTITUT FUR PHARMAZEUTISCHE TECHNOLOGIE), SAF-1 (SYNTEX ADJUVANT FORMULATION CHIRON), Stearyl tyrosine (BIOCHEM THERAPEUTIC INC.), Theramidea (IMMUNO THERAPEUTICS INC.), Threonyl-MDP (CHIRON), FREUNDS adjuvant (complete or incomplete), aluminum hydroxide, dimethyldioctadecyl-ammonium bromide, Adjuvax (ALPHA-BETA TECHNOLOGY), Inject Alum (PIERCE), Monophosphoryl Lipid A (RIBI IMMUNOCHEM RESEARCH), MPL+TDM (RIBI IMMUNOCHEM RESEARCH), Titermax (CYTRX), QS21, t Ribi Adjuvant System, TiterMaxGold, QS21, Adjumer, Calcitrol, CTB, LT (E. coli toxin), LPS (lipopolysaccharide), Avridine, the CpG sequences (Singh et al., 1999 Singh, M. and Hagum, D., Nature Biotechnology 1999 17: 1075-81) toxins, toxoids, glycoproteins, lipids, glycolipids, bacterial cell walls, subunits (bacterial or viral), carbohydrate moieties (mono-, di, tri-, tetra-, oligo- and polysaccharide), or saponins. Combinations of various adjuvants may be used with the antigen to prepare the immunogen formulations. Adjuvants administered parentally or for the induction of mucosal immunity may also be used.

The present invention further contemplates isolated and/or purified antibody, antibody fragment or derivative thereof able to bind to the at least one folded sequence of a S. aureus adhesion of the invention, or to the sequence having 80% or more sequence identity to said folded sequence of a S. aureus adhesion of the invention, expressed on the cell surface of an inactivated recombinant non-pathogenic bacterium.

As used herein, an “antibody” is a protein molecule that reacts with a specific antigenic determinant or epitope and belongs to one or five distinct classes based on structural properties: IgA, IgD, IgE, IgG and IgM. The antibody may be a polyclonal (e.g. a polyclonal serum) or a monoclonal antibody, including but not limited to fully assembled antibody, single chain antibody, antibody fragment, and chimeric antibody, humanized antibody as long as these molecules are still biologically active and still bind to one folded sequence of a S. aureus adhesin of the invention. Preferably the antibody is a monoclonal antibody. Preferably also the monoclonal antibody will be selected from the group comprising the IgGI, IgG2, IgG2a, IgG2b, IgG3 and IgG4 or a combination thereof. Most preferably, the monoclonal antibody is selected from the group comprising the IgGI, IgG2, IgG2a, and IgG2b, or a combination thereof.

A typical antibody is composed of two immunoglobulin (Ig) heavy chains and two Ig light chains. Several different types of heavy chain exist that define the class or isotype of an antibody. These heavy chain types vary between different animals. All heavy chains contain a series of immunoglobulin domains, usually with one variable (VH) domain that is important for binding antigen and several constant (CH) domains. Each light chain is composed of two tandem immunoglobulin domains: one constant (CL) domain and one variable domain (VL) that is important for antigen binding.

The term “isolated”, when used as a modifier of an antibody of the invention means that the antibody is made by the hand of man or is separated, completely or at least in part, from their naturally occurring in vivo environment. Generally, isolated antibodies are substantially free of one or more materials with which they normally associate with in nature, for example, one or more protein. The term “isolated” does not exclude alternative physical forms of the antibodies, such as multimers/oligomers, modifications (e.g., phosphorylation, glycosylation, lipidation) or derivatized forms, or forms expressed in host cells produced by the hand of man

An “isolated” antibody can also be “substantially pure” or “purified” when free of most or all of the materials with which it typically associates with in nature. Thus, an isolated antibody that also is substantially pure or purified does not include polypeptides or polynucleotides present among millions of other sequences, such as antibodies of an antibody library or nucleic acids in a genomic or cDNA library.

Antibodies used in the present invention are not limited to whole antibody molecules and may be antibody fragments or derivatives as long as they are able to bind to the at least one folded sequence of a S. aureus adhesin expressed on the cell surface of an inactivated recombinant non-pathogenic bacterium and that they specifically recognize said folded sequence of a S. aureus adhesin.

Examples of isolated and/or purified antibody fragment or derivative thereof are selected amongst the group comprising a Fab-fragment, a F(ab2)′-fragment, a single-chain antibody, a chimeric antibody, a CDR-grafted antibody, a bivalent antibody-construct, a humanized antibody, a synthetic antibody, a chemically modified derivative thereof, a multispecific antibody, a diabody, a scFv-fragment; a dsFv-fragment, a labeled antibody, or another type of recombinant antibody. Specifically, an antibody fragment is synthesized by treating the antibody with an enzyme such as papain or pepsin, or genes encoding these antibody fragments are constructed, and expressed by appropriate host cells as known to the skilled artisan.

Yet another concern of the present invention is to provide an expression vector comprising at least one isolated and/or purified nucleic acid sequence encoding for at least one folded sequence of a S. aureus adhesin, or a sequence having 80% or more sequence identity to said folded sequence of a S. aureus adhesin. Preferably the nucleic acid molecule sequence encoding a peptide of the invention is DNA.

As used herein, “vector”, “plasmid” and “expression vector” are used interchangeably, as the plasmid is the most commonly used vector form.

The vector may further comprise a promoter operably linked to the sequence encoding a folded sequence of a S. aureus adhesin. This means that the linked isolated and purified DNA sequence encoding the peptide of the present invention is under control of a suitable regulatory sequence which allows expression, i.e. transcription and translation of the inserted isolated and purified DNA sequence.

As used herein, the term “promoter” designates any additional regulatory sequences as known in the art e.g. a promoter and/or an enhancer, polyadenylation sites and splice junctions usually employed for the expression of the polypeptide or may include additionally one or more separate targeting sequences and may optionally encode a selectable marker. Promoters which can be used provided that such promoters are compatible with the host cell are e.g promoters obtained from the genomes of viruses such as polyoma virus, adenovirus (such as Adenovirus 2), papilloma virus (such as bovine papilloma virus), avian sarcoma virus, cytomegalovirus (such as murine or human cytomegalovirus immediate early promoter), a retrovirus, hepatitis-B virus, and Simian Virus 40 (such as SV 40 early and late promoters) or promoters obtained from heterologous mammalian promoters, such as the actin promoter or an immunoglobulin promoter or heat shock promoters.

Enhancers, which can be used, are e.g. enhancer sequences known from mammalian genes (globin, elastase, albumin, alpha-fetoprotein, and insulin) or enhancer from a eukaryotic cell virus. e.g. the SV40 enhancer, the cytomegalovirus early promoter enhancer, the polyoma, and adenovirus enhancers.

Useful expression vectors, for example, may consist of segments of chromosomal, non-chromosomal and synthetic DNA sequences. Suitable vectors include derivatives of SV40 and known bacterial plasmids, phage DNAs, yeast plasm ids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. Most preferably the expression vector is a lactococcal plasmid. More preferably, the lactococcal plasmid is pOri23.

Also provided is a method for treating and/or preventing infections and diseases caused by S. aureus, in a subject in need thereof, comprising administering a pharmaceutically effective amount of an immunogenic composition according to the invention.

Usually, infections and diseases caused by S. aureus are selected among the non limiting group comprising IE, intravascular and intravascular device infections, bloodstream infections, deep-seated abscesses, osteomyelitis, infection of prosthetic materials, and skin and soft tissue infections. Examples of diseases caused by S. aureus are folliculitis, furunculosis, erysipelas, deep-seated abscesses, osteomyelitis, pneumonia, sepsis, and infective endocarditis (IE).

Also envisioned is a method of inducing active immunity against a S. aureus infection in a subject in need thereof, comprising administering to said subject in need thereof i) an immunogenic composition comprising an inactivated recombinant non-pathogenic bacterium, or a part thereof, expressing on its cell surface, at least one folded sequence of a S. aureus adhesin or ii) a vaccine of the invention.

Also encompassed in the present invention is a method of inducing passive immunity against a S. aureus infection in a subject in need thereof, comprising administering to said subject in need thereof an isolated and/or purified antibody, antibody fragment or derivative thereof of the invention.

### EXAMPLES

### Example 1

### Materials and Methods

### 1.1 Plasmid Constructs and Bacterial Strains

The immunization protocol in this study was done using the recombinant strain of the non-pathogenic L. lactis subsp. cremoris 1363 expressing individual S. aureus ClfA, described elsewhere (Piroth et al., 2008; Que et al., 2000; Que et al., 2001) or S. aureus ClfA/FnBPA(CD). The S. aureus Newman ClfA gene was inserted in the lactococcal plasmid pOri23 together with an erythromycin resistance determinant as described by Que et al (Que et al., 2000). In the strain L. lactis ClfA/FnBPA(CD) S. aureus Newman ClfA gene was expressed as described above, and the CD domain of the S. aureus 8325 FnBPA gene was inserted in lactococcal plasmid pOri23 together with an kanamycin resistance determinant (Elonora Widmer MD/PhD thesis). The L. lactis plL253, containing the lactococcal plasmid pOri23 expressing only the erythromycin resistance determinant, and expressing no pathogenic factors, was used as the control mutant strain. All lactococci were grown at 30° C. without shaking in M17 medium (Oxoid) or on M17 agar plates supplemented with 0.5% glucose and 5 μg/m1 erythromycin (plus kanamycin when appropriate).

The well-described S. aureus strain Newman (i.e. methicillin-susceptible S. aureus) S. aureus strain P8 (i.e. methicillin-resistant S. aureus) (Entenza et al., 2001) was used in the animal model of endocarditis. The S. aureus bacterial isolates was grown at 37° C. in tryptic soy broth (Difco).

All the bacterial stocks were kept at −80° C. in liquid medium supplemented with 20% (vol/vol) of glycerol.

### 1.2 Immunization Protocol

(i) Animals. Four to six-week females (100 g of weight) Wistar Han rats were purchased from Charles River, France. The rats were supplied with water and food ad libitum, and randomly allocated to 6 treatment groups as follows: (a) Control, (b) Freund's adjuvant (Sigma), (c) L. lactis plL253 and (d) L. lactis ClfA, L. lactis FnBPA or L. lactis ClfA/FnBPA(CD). All animal experiments were carried out according to Swiss regulations (authorization 879.8).
(ii) Preparation of bacterial vaccine. The inactivated L. lactis vaccine was prepared as follows. L. lactis strains carrying either the empty plasmid pOri23, or the plasmid expressing ClfA or ClfA/FnBPA(CD) were cultured overnight at 30° C. without shaking in M17 medium (Oxoid), harvested by centrifugation, resuspended in sterilized PBS, and adjusted to 1×10⁸ CFU/ml. The bacteria were then inactivated during 60 min under U.V. Then, the bacteria were emulsified at a 1:1 ratio in Freund's adjuvant. The first immunization was done with Freund's complete adjuvant, and the subsequent with Freund's incomplete adjuvant. The control group was immunized with PBS, and the Freund's adjuvant group with the adjuvant emulsified at a 1:1 ratio in PBS.
(iii) Vaccination schedule. The rats were immunized at 2-week intervals (days 0, 14 and 28). Three hundred microlitres of the preparations were injected intra-peritoneal to the respective groups. Blood samples were collected on days 7, 21 and 35; and the sera were harvested and stored at −80° C. to posterior in vitro analysis (Gong et al., 2010).

### 1.3. Animal Model of Endocarditis

Catheter-induced aortic vegetations were produced according to the method of Heraief et at (Héraïef et al., 1982) Insertion of an intravenous (i.v.) line in the jugular vein and connection to a programmable infusion pump (Pump 44; Harvard Apparatus, Inc., South Natick, Mass.) to deliver the inocula was performed as described (Fluckiger et al., 1994; Pea et al., 2011) on the day 40 of the vaccination schedule. Bacterial inocula were prepared from overnight cultures. Microorganisms were recovered by centrifugation, washed and adjusted to the desired inoculum size in saline. The inoculum size was confirmed by colony counts on blood agar plates. Animals were inoculated 24 h after catheterization, via the infusion pump, with 1 ml of 10⁴ CFU(S. aureus Newman) or 106 CFU (S. aureus P8) progressively delivered at a pace of 0.0017 ml/min over 10 h in order to produce a low-grade of bacteremia (Veloso et al. 2011).

The traditional i.v. bolus inoculation (10⁴ CFU/ml) provoking transient high-grade bacteremia was also performed. Rats were sacrificed 24 h after the end of inoculation. Quantitative valve cultures were performed as previously described (Fluckiger et al., 1994) This method permitted the detection of 2 log 10 CFU/g of vegetation.

### 1.4. Statistical Analysis

Statistical analyses were performed using GraphPad software (GraphPad Software, Inc., USA). The rates of valve infections of the various groups were compared by the x2 test. P<0.05 was considered to be statistically significant.

### 1.5. Trypsin Shaving Protocol (Ythier et al. 2012)

In brief, bacteria were grown in 300 ml liquid cultures in the different media described above. At various times of the logarithmic or stationary growth phases, samples (between 10 and 100 ml depending on the cell density) were removed, immediately chilled at 4° C., and harvested by centrifugation. Pellets were washed three times with ice-cold phosphate-buffered saline (PBS) and finally re-suspended in 1 ml of the same buffer. To allow semi-quantitative comparisons between the proteomes of different samples, cell concentrations were adjusted to 1×10⁹ bacteria/ml in all samples. Cell counts were validated by optical microscopy (Neubauer cell) and viable colony counts on nutrient agar. There were <0.5 log 10 differences between the Neubauer cell and viable counts, indicating that the large majority of cells were alive. Samples were then shaved for 1 h with 1 μg/ml (final concentration) of trypsin (Promega, Madison, Wis.) at 37° C., after which they were chilled at 4° C. and bacterial cells removed by centrifugation for 10 min at 4000 rpm and 4° C. Supernatants containing trypsin-shaved peptides were filtered (0.22 μm) and freeze-dried until further use.

### Example 2

### Results

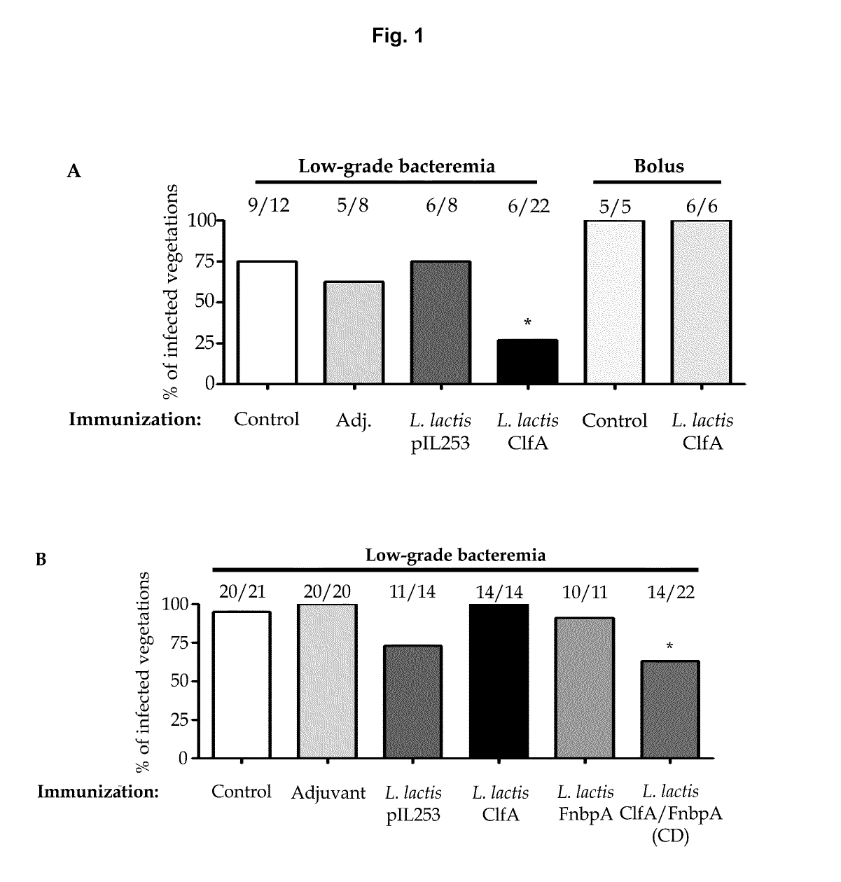

The efficacy of the immunization with L. lactis ClfA or L. lactis ClfA/FnBPA(CD) against the S. aureus induced experimental endocarditis due to low-grade bacteremia was compared to the different control groups. The results of the infectivity rate are shown in FIG. 1.

In FIG. 1A the proportion of infection in the group immunized with L. lactis ClfA (6/22; 27.2%) was significantly lower than in the control groups PBS (9/12; 75%), Adj. (5/8; 62.5%) and L. lactis pIL253 (6/8; 75%) (χ² test; P<0.05), in the case of low-grade bacteremia experimental endocarditis induced by S. aureus Newman. These results confirm the protective effect of the immunization using the non-pathogenic L. lactis expressing heterologously the staphylococcal adhesin ClfA. In contrast, when the same immunization schedule was used in animals exposed to high-grade bacteremia experimental endocarditis, the protective effect was not observed.

In FIG. 1B the proportion of infection in the group immunized with L. lactis ClfA/FnBPA(CD) (14/22; 63.6%) was significantly lower (P<0.05) than in the control groups PBS (20/21; 95.2%), Adj. (20/20; 100%) but not for L. lactis pIL253 (11/15; 73.3%; χ² test; P=0.53). These results demonstrate a diminution in the infection after the effect of the immunization using the non-pathogenic L. lactis expressing heterologously the staphylococcal adhesins ClfA/FnBPA (CD).

### REFERENCES

Broughan, J., Anderson, R., & Anderson, A. S. (2011). Strategies for and advances in the development of Staphylococcus aureus prophylactic vaccines. Expert review of vaccines, 10(5), 695-708.
Entenza, J. M., Que, Y. A., Vouillamoz, J., Glauser, M. P., & Moreillon, P. (2001). Efficacies of moxifloxacin, ciprofloxacin, and vancomycin against experimental endocarditis due to methicillin-resistant Staphylococcus aureus expressing various degrees of ciprofloxacin resistance. Antimicrobial agents and chemotherapy, 45(11), 3076-83.
Fluckiger, U., Moreillon, P., Blaser, L. J., Bickle, M., Glauser, M. P., & Francioli, P. (1994). Simulation of Amoxicillin Pharmacokinetics in Humans for the Prevention of Streptococcal Endocarditis in Rats. Antimicrobial agents and chemotherapy, 38(12), 2846-2849.
Gong, R., Hu, C., Xu, H., Guo, A., Chen, H., Zhang, G., & Shi, L. (2010). Evaluation of clumping factor A binding region A in a subunit vaccine against Staphylococcus aureus-induced mastitis in mice. Clinical and vaccine immunology: CVI, 17(11), 1746-52.
Héraïef E., Glauser, M. P., & Freedman, L. R. (1982). Natural history of aortic valve endocarditis in rats. Infection and immunity, 37(1), 127-31.
Kuhn, G., Francioli, P., & Blanc, D. S. (2007). Double-locus sequence typing using clfB and spa, a fast and simple method for epidemiological typing of methicillin-resistant Staphylococcus aureus. Journal of clinical microbiology, 45(1), 54-62.
Mcdevitt, D., Francois, P. F., Vaudaux, P., & Foster, T. J. (1994). Molecular characterization of the clumping factor (fibrinogen receptor) of Staphylococcus aureus. Mol. Microbiol, 11(2), 237-248.
Moreillon, P., Entenza, J. M., Francioli, P., McDevitt, D., Foster, T. J., & Vaudaux, P. (1995). Role of Staphylococcus aureus Coagulase and Clumping Factor in Pathogenesis of Experimental Endocarditis. Infection and immunity, 63(12), 4738-4743.
Patti, J. M., Allen, B. L., Mcgavin, M. J., & Hook, M. (1994). MSCRAMM-Mediated Adherence of Microorganisms to Host Tissues. Ann. Rev. Microbiol., 48, 585-617.
Pea, F., Cojutti, P., Petrosillo, N., Furlanut, M., Entenza, J. M., Veloso, T. R., Vouillamoz, J., et al. (2011). Continuous infusion may improve the efficacy of vancomycin in treatment of experimental endocarditis due to heterogeneous vancomycin-intermediate Staphylococcus aureus. Antimicrobial agents and chemotherapy, 55(9), 4496;
Piroth, L., Que, Y., Widmer, E., Panchaud, A., & Moreillon, P. (2008). The Fibrinogen- and Fibronectin-Binding Domains of Staphylococcus aureus Fibronectin-Binding Protein A Synergistically Promote Endothelial Invasion and Experimental Endocarditis. Infection and immunity, 76(8), 3824-3831.
Que, Y, & Moreillon, P. (2011). Infective endocarditis. The Lancet, 363,139-149. Nature Publishing Group.
Que, Y. A., Haefliger, J. A., Francioli, P., & Moreillon, P. (2000). Expression of Staphylococcus aureus clumping factor A in Lactococcus lactis subsp. cremoris using a new shuttle vector. Infection and immunity, 68(6), 3516-22.
Que, Y. A., & Moreillon, P. (2009). Principles and Practice in Infectious Diseases. (G. Mandell, R. Douglas, & J. Bennett, Eds.) (p. Chapter 195). New York: Elsevier Inc.
Que, Yok-Ai, Franc, P., Haefliger, J., Vaudaux, P., & Moreillon, P. (2001). Reassessing the Role of Staphylococcus aureus Clumping Factor and Fibronectin-Binding Protein by Expression in Lactococcus lactis. Infection and immunity, 69(10), 6296-6302.
Que, Yok-Ai, Haefliger, J., Piroth, L., Widmer, E., Sinha, B., Herrmann, M., Francioli, P., et al. (2005). Fibrinogen and fibronectin binding cooperate for valve infection and invasion in Staphylococcus aureus experimental endocarditis. JEM, 201(610), 1627-1635.
Risley, A. L., Loughman, A., Cywes-Bentley, C., Foster, T. J., & Lee, J. C. (2007). Capsular polysaccharide masks clumping factor A-mediated adherence of Staphylococcus aureus to fibrinogen and platelets. The Journal of infectious diseases, 196(6), 919-27.
Scarpa, M., Piccinini, R., Brun, P., Grillo, A., Palù, G., Mengoli, C., Daprà, V., et al. (2010). Relationship between virulence factor genes in bovine Staphylococcus aureus subclinical mastitis isolates and binding to anti-adhesin antibodies. The Journal of dairy research, 77(2), 159-67.
Shinefield, H., Black, S., Fattom, A., Horwith, G., Rasgon, S., Ordonez, J., Yeoh, H., et al. (2002). Use of a Staphylococcus aureus conjugate vaccine in patients receiving hemodialysis. The New England journal of medicine, 346(7), 491-6.
Veloso, T. R., Amiguet, M., Rousson, V., Giddey, M., Vouillamoz, J., Moreillon, P., & Entenza, J. M. (2011). Induction of experimental endocarditis by continuous low-grade bacteremia mimicking spontaneous bacteremia in humans. Infection and immunity, 79(5), 2006-11.
Wells, J. M., & Mercenier, A. (2008). Mucosal delivery of therapeutic and prophylactic molecules using lactic acid bacteria. Nature reviews. Microbiology, 6(5), 349-62.
Ythier, M., Resch, G., Waridel, P., Panchaud, A., Gfeller, A., Majcherczyk, P., Quadroni, M., et al. (2012). Proteomic and transcriptomic profiling of Staphylococcus aureus surface LPXTG-proteins: correlation with agr genotypes and adherence phenotypes. Molecular & cellular proteomics: MCP.

1. An immunogenic composition comprising an inactivated recombinant non-pathogenic bacterium, or a part thereof, expressing on its cell surface at least one folded sequence of a S. aureus adhesin, or a sequence having 80% or more sequence identity to said folded sequence of a S. aureus adhesin, wherein said at least one folded sequence of a S. aureus adhesin, or sequence having 80% or more sequence identity to said folded sequence of a S. aureus adhesin, is entirely accessible to trypsin digestion.

2. The immunogenic composition of claim 1, wherein the trypsin digestion of said at least one folded sequence of a S. aureus adhesin, or of the sequence having 80% or more sequence identity to said folded sequence of a S. aureus adhesin, releases a digestion profile upon trypsin digestion which is different from a digestion profile upon trypsin digestion of said adhesin expressed on the surface of S. aureus.

3. The immunogenic composition of claim 1, wherein the part of the inactivated recombinant non-pathogenic bacterium consists of an isolated and/or purified cell wall.

4. The immunogenic composition of claim 1, wherein the at least one folded sequence of a S. aureus adhesin is selected from the group consisting of fibrinogen-binding protein A (clumping factor A ClfA), fibrinogen-binding protein B (ClfB), fibronectin-binding protein A (FnPBA) and fibronectin-binding protein B (FnBPB), collagen-binding protein (Cna) and protein A (Spa), Serine-aspartate repeat protein C, D and E (SdrC-E), Plasmin-sensitive protein (Pls), Factor affecting methicillin resistance in the presence of Triton X-100 (FmtB), and surface protein A-K (SasA-K), or a combination thereof.

5. The immunogenic composition of claim 4, wherein the at least one folded sequence of a S. aureus adhesin consists of the fibrinogen-binding protein A (clumping factor A (ClfA)) and/or fibronectin-binding protein A (FnPBA).

6. The immunogenic composition of claim 1, wherein the inactivated recombinant non-pathogenic bacterium is the L. lactis subspecies cremoris 1363.

7. The immunogenic composition of claim 4, wherein the sequence of S. aureus fibrinogen-binding protein A (clumping factor A (ClfA)) is as set forth in SEQ ID No.1.

8. The immunogenic composition of claim 1, wherein the S. aureus fibrinogen-binding protein A (clumping factor A (ClfA)) releases a digestion profile of 9 peptides upon trypsin digestion.

9. The immunogenic composition of claim 1, wherein the S. aureus fibronectin-binding protein A (FnPBA) releases a digestion profile of 23 peptides upon trypsin digestion.

10. The immunogenic composition of claim 1, wherein the inactivated recombinant non-pathogenic bacterium is UV inactivated.

11. A vaccine comprising an immunogenic composition of claim 1 in an immunologically acceptable carrier and/or diluent.

12. The vaccine of claim 11, wherein the immunologically acceptable carrier is selected from the group consisting of polysaccharide materials forming hydrogels and vesicular carriers.

13. The vaccine of claim 12, wherein the vesicular carriers are selected from the group consisting of bacterial ghosts, liposomes, niosomes, transfersomes, and ethosomes.

14. The vaccine of claim 11, further comprising an adjuvant.

15. (canceled)

16. The method of claim 19, wherein the infection or disease caused by S. aureus is selected from the group consisting of IE, intravascular and intravascular device infections, bloodstream infections, deep-seated abscesses, osteomyelitis, infection of prosthetic materials, and skin and soft tissue infections.

17. An isolated and/or purified antibody, antibody fragment or derivative thereof able to bind to the at least one folded sequence of a S. aureus adhesin, or to a sequence having 80% or more sequence identity to said folded sequence of a S. aureus adhesin, expressed on the cell surface of an inactivated recombinant non-pathogenic bacterium.

18. An expression vector comprising an isolated and/or purified nucleic acid sequence encoding for at least one folded sequence of a S. aureus adhesin, or a sequence having 80% or more sequence identity to said folded sequence of a S. aureus adhesin.

19. A method for treating and/or preventing an infection or disease caused by S. aureus, in a subject in need thereof, comprising administering a pharmaceutically effective amount of an immunogenic composition of claim 1.

20. A method for inducing active immunity against an infection or disease caused by S. aureus in a subject in need thereof, comprising administering to said subject in need thereof i) an immunogenic composition of claim 1 or ii) a vaccine of claim 11.

21. A method for inducing passive immunity against an infection or disease caused by S. aureus in a subject in need thereof, comprising administering to said subject in need thereof an isolated and/or purified antibody, antibody fragment or derivative thereof of claim 17.

22. The method of claim 20, wherein the infection or disease caused by S. aureus is selected from the group consisting of IE, intravascular and intravascular device infections, bloodstream infections, deep-seated abscesses, osteomyelitis, infection of prosthetic materials, and skin and soft tissue infections.

23. The method of claim 21, wherein the infection or disease caused by S. aureus is selected from the group consisting of IE, intravascular and intravascular device infections, bloodstream infections, deep-seated abscesses, osteomyelitis, infection of prosthetic materials, and skin and soft tissue infections.
